(19) United States
(12) Patent Application Publication
(10) Pub. No.: US 2004/0234457 A1
(43) Pub. Date: Nov. 25, 2004

(54) Methods of preventing and treating SARS using low pH respiratory tract compositions

(75) Inventors: Paul John Rennie, Godalming (GB); Shane Michael De La Harpe, Liskeard (GB); Jayant Ekanth Khanolkar, Surbiton (GB); Benoit Maurice Mullet, Chartres (FR)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company, Cincinnati, OH

(21) Appl. No.: 10/770,138

(22) Filed: Feb. 2, 2004

Related U.S. Application Data
(63) Continuation-in-part of application No. 09/692,634, filed on Oct. 19, 2000.
Application No. 09/692,634 is a continuation-in-part of application No. 09/421,131, filed on Oct. 19, 1999, now abandoned.

(51) Int. Cl. A61L 9/04; A61K 9/14; A61K 33/24

(52) U.S. Cl. 424/46

(57) ABSTRACT

The present invention is directed to methods of preventing and treating Severe Acute Respiratory Syndrome (SARS) by the administration of respiratory tract compositions having low pH values. The respiratory tract compositions have a pH of from about 3.0 to about 5.5, and are preferably administered to areas of the respiratory tract such as the nasal cavity to effectively prevent and treat respiratory tract viral infections, particularly SARS. The respiratory tract compositions comprise an organic acid in combination with a metal compound.

### CROSS REFERENCE TO RELATED APPLICATION

This is a continuation-in-part of application Ser. No. 09/692,634 (P&G Case 8308), filed on Oct. 19, 2000, which is a continuation-in-part of application Ser. No. 09/421,131 (P&G Case 7831), filed on Oct. 19, 1999.

### FIELD OF THE INVENTION

The present invention is directed to methods of preventing and treating Severe A cute Respiratory Syndrome (hereinafter SARS) by the administration of respiratory tract compositions having low pH values. In particular, the present invention is directed to methods of preventing and treating viruses associated with SARS by administering respiratory tract compositions having a pH of from about 3.0 to about 5.5 to areas of the upper respiratory tract.

### BACKGROUND OF THE INVENTION

SARS is a respiratory tract viral infection that is believed to be the result of viral infection caused by a family of viruses known as coronaviruses, viruses typically associated with the common cold. Symptoms of SARS are similar to cold and influenza-like symptoms, for example, an individual infected with SARS typically exhibits symptoms such as high fever, body aches, headache, dry cough, chills, fatigue or malaise, diarrhea, dyspnea (shortness of breath), and hypoxaemia (low blood oxygen concentration).

To treat individuals infected with SARS, remedies to treat the upper and/or lower respiratory tract areas have been suggested. Suitable treatment methods include vaccinations against SARS, and the administration of antibiotics or antiviral drugs such as ribavirin, tetracycline, erythromycin, and corticosteroids (e.g., methylprednisolone). Diagnostic testing to mitigate SARS has also been suggested, wherein suitable diagnostic testing include serological testing for anti-coronavirus antibodies, and molecular testing such as transcriptase-polymerase chain reaction (RT-PCR) for specific coronavirus RNA. However, treatment methods and ways to prevent SARS are still being sought and studied.

Although little is known about the specific causes and treatment methods related to SARS, there exist published documents that describe methods of treating symptoms associated with SARS. For example, U.S. patent application Publication No. 2004/0009245 discloses methods and apparatus to prevent, treat, and cure infections of the respiratory pathogens causing SARS, wherein the methods and apparatus employ the inhalation of concentrated vapors from botanical essential oils such as essential oils from Eucalyptus globules, Melaleuca alternifolia, Eucalyptus citriodora, and Eucalyptus radiate.

Other disclosures of methods to treat SARS related illnesses include the method disclosed in WO 03/094902. This disclosure is related to surface treatment of SARS-infected lungs by injecting sterilizing liquid into lung lobes, wherein a suitable sterilizer is identified as ozone and the liquid is described as Per fluro chemicals (PFC).

Another disclosure of a method to treat SARS related illnesses include the therapeutic method for treating biological diseases as disclosed in WO 03/086408. This disclosure is directed to a therapeutic method for treating biological diseases wherein the method includes the administration of an effective amount of a suitable antibiotic agent, antifungal agent or antiviral agent in conjunction with A_{2A} adenosine receptor agonist. WO 03/086408 further discloses that the A_{2A} adenosine receptor agonist can be used alone to treat inflammation associated with certain viruses such as those that cause SARS.

Still yet another disclosure of a method to treat SARS related illnesses include the method disclosed in WO 03/105775 which is related to the administration of a powder formulation to treat respiratory diseases or conditions such as SARS. The powder formulation disclosed in WO 03/105775 comprises dehydrate dehydroepiandrosterone covalently bound to a sulfate. WO 03/105775 further discloses that the powder formulation can be delivered through the respiratory tract using delivery means such as a nebulizer, a dry powder inhaler, an insufflator, an aerosol, or a spray generator.

Still yet another disclosure of a method to treat SARS related illnesses include the administration of formulations disclosed in WO 03/092654. WO 03/092654 discloses formulations for pulmonary delivery to treat or reduce the infectivity of diseases such as SARS, wherein the formulations comprise a material such as a surfactant that alters surface tension and surface elasticity of lung mucus lining fluid.

In view of the limited disclosures of attempts to prevent and treat symptoms associated with SARS, the need still exists for highly effective methods in the prevention and treatment of SARS related illnesses. It has been found that the administration of respiratory tract compositions having a pH of from about 3.0 to about 5.5 are effective in creating an environment that is hostile to SARS-associated viruses such as the coronavirus. Such an environment deters viruses from infecting areas of the respiratory tract that are conducive to SARS viral infections. The low pH respiratory tract compositions are also suitable for method applications of reducing or eliminating the possibility of acquisition of SARS viruses when confronted with a high-risk public environment including schools, office buildings, hotels, and airports.

### SUMMARY OF THE INVENTION

The present invention is directed to methods of preventing and treating SARS by administering respiratory tract compositions having a pH of from about 3.0 to about 5.5 to areas of the upper respiratory tract. A particular respiratory tract composition includes a respiratory tract composition that has a pH of from about 3.0 to about 5.5, and that comprises an organic acid in combination with a metal compound. The respiratory tract composition preferably comprises an optional mucoadhesive polymer in addition to the organic acid and metal compound.

The respiratory tract composition is preferably a respiratory tract composition that is administered intranasally to areas of the respiratory tract. Respiratory tract compositions in the form of nasal respiratory tract compositions have been found to effectively contact mucosal tissues and fluids to thus create an environment that is hostile to SARS-associated viruses. As used herein, mucosal tissues and fluids include the mucosa of the nose, mouth, tongue, and throat. T he nasal respiratory tract compositions can be administered in the form of droppers, pump sprayers, pressurized sprayers, atomizers, air inhalation devices, nasal irrigations, and the like. The nasal respiratory tract compositions are especially effective when the compositions are administered using nasal sprays that have a pH of from about 3.0 to about 5.5, and that comprise an organic acid defined herein in combination with a metal compound defined herein.

The respiratory tract compositions can be used alone or in combination with other known or otherwise effective antiviral compositions to prevent and treat symptoms associated with SARS. For example, it is contemplated that the respiratory tract compositions can be used alone or in combination with an antiviral composition such as the low pH antimicrobial compositions that comprise an organic acid in combination with a short-chain anionic surfactant and that are described in U.S. patent application Publication No. 2003/0235550, which descriptions are incorporated herein by reference.

### DETAILED DESCRIPTION OF THE INVENTION

The methods of the present invention involve administering respiratory tract compositions to the upper respiratory tract areas, particularly to areas such as the nasal cavity. The respiratory tract compositions are effective in the prevention and treatment of SARS-associated viruses in addition to preventing and treating any known or otherwise effective viruses that result in symptoms associated with SARS, the common cold, and influenza. The respiratory tract compositions are typically administered using a pharmaceutically acceptable vehicle sometimes referred to as carrier systems.

The respiratory tract compositions are especially administered to prevent and treat symptoms associated with SARS, however, the compositions are effective in preventing and treating cold and influenza-like symptoms. As used herein “SARS, cold, and influenza-like symptoms” refer to symptoms typically associated with respiratory tract viral infections. These symptoms include, but are not limited to, nasal congestion, chest congestion, sneezing, rhinorrhea, fatigue or malaise, coughing, fever, chills, body aches, sore throat, headache, dry cough, diarrhea, dyspnea (shortness of breath), hypoxaemia (low blood oxygen concentration) and other known SARS, cold, and influenza-like symptoms.

The term “SARS-associated viruses” as used herein typically refers to those viruses associated with Coronaviruses, including any known and yet to be discovered species of the Coronavirus. The Coronavirus is also known as a casual agent of cold and influenza-like symptoms. Accordingly, the methods of the present invention are effective in the prevention and treatment of viruses including Rhinovirus, Myxovirus (Influenza virus), Paramyxovirus (Parainfluenza virus), Respiratory Syncytial virus, Adenovirus, and Coronavirus.

The term “upper respiratory tract” as used herein refers to the areas of the nose, mouth, tongue, and throat, including the mucosal membranes of the nose, mouth, tongue, and throat.

The term “pharmaceutically acceptable vehicle” refers to any solid, liquid or gas combined with components of the respiratory tract compositions of the present invention to deliver the components to the upper respiratory tract of a user. These vehicles are generally regarded as safe for internal and/or topical use by mammals including humans and animals. The “pharmaceutically acceptable vehicles” are also known as carrier systems.

The method of the present invention includes the administration of respiratory tract compositions that can comprise, consist of, or consist essentially of the elements and limitations of the invention described herein, as well as any of the additional or optional ingredients, components, or limitations described herein.

All percentages, parts and ratios are by weight of the respiratory tract compositions, unless otherwise specified. All such weights as they pertain to listed ingredients are based on the specific ingredient level and, therefore, do not include carriers or by-products that may be included in commercially available materials, unless otherwise specified.

All documents cited herein, including publications, patent applications, and issued patents mentioned herein, are, in relevant part, incorporated herein by reference. Citation of any document is not an admission regarding any determination as to its availability as prior art to the present invention.

### Organic Acid

The methods of the present invention include the administration of respiratory tract compositions that comprise an organic acid that provides for an environment that is hostile to viruses known to contribute to symptoms associated with respiratory tract viral infections, particularly SARS.

The organic acid suitable for use herein can be included in the respiratory tract compositions as an individual organic acid or as a combination of organic acids, provided that the total organic acid concentration ranges from about 0.01% to about 20%, preferably from about 0.05% to about 10%, more preferably from about 0.10% to about 5%, by weight of the composition.

The organic acid suitable for use herein has a dissociation constant (pKa) of from about 3.0 to about 5.5 which provides for buffering capacity of the respiratory tract compositions. For respiratory tract compositions of the present invention that are administered intranasally, the buffering capacity provides for a surface pH of the tissue treated in the nasal cavities or turbinates of from about 3.0 to about 5.5 to create an environment that is hostile to viruses associated with respiratory tract viral infections.

Nonlimiting examples of an organic acid suitable for use herein include pyroglutamic acid (PCA), ascorbic acid, monocarboxylic acids, dicarboxylic acids, tricarboxylic acids, and mixtures thereof. Specific nonlimiting examples of suitable monocarboxylic, dicarboxylic, or tricarboxylic acids include salicylic, fumaric, benzoic, glutaric, lactic, citric, malonic, acetic, glycolic, malic, adipic, succinic, aspartic, phthalic, tartaric, glutamic, gluconic, and mixtures thereof.

It has been found that a combination of pyroglutamic acid and a monocarboxylic, dicarboxylic acid, or tricarboxylic acid defined herein is highly effective in providing for increased buffering capacity to result in respiratory tract compositions that can be administered intranasally to prevent and treat symptoms related to respiratory viral infections such as SARS.

When the respiratory tract compositions comprise pyroglutamic acid, the pyroglutamic acid is typically included at concentrations ranging from about 0.01% to about 20%, preferably from about 0.1% to about 10%, more preferably from about 0.25% to about 8%, most preferably from about 0. 1% to about 5%, by weight of the composition.

Nonlimitimg specific examples of pyroglutamic acid suitable for use in the respiratory tract compositions of the present invention include those pyroglutamic acid compounds collectively referred to as stereoisomers and tautomers of pyroglutamic acid. Pyroglutamic acid, which is also referred to as pyrrolidone carboxylic acid has two stereoisomers (D and L) and each are preferred for use herein. Pharmaceutically acceptable salts of pyroglutamic acid are also suitable for use herein.

The D stereoisomer of pyroglutamic acid is also known by the following names: D-Proline, 5-oxo-(+)-2-Pyrrolidone-5-carboxylic acid, (+)-Pyroglutamic acid, (R)-2-Pyrrolidone-5-carboxylic acid, 5-Oxo-D-proline, D-2-Pyrrolidone-5-carboxylic acid, D-Pyroglutamic acid, D-Pyrrolidinonecarboxylic acid, and D-Pyrrolidonecarboxylic acid.

The L stereoisomer of pyroglutamic acid is also known by the following names: L-Proline, 5-oxo-(−)-2-Pyrrolidone-5-carboxylic acid, (−)-Pyroglutamic acid, (5S)-2-Oxopyrrolidine-5-carboxylic acid, (S)-(−)-2-Pyrrolidone-5-carboxylic acid, (S)-2-Pyrrolidone-5-carboxylic acid, (S)-5-Oxo-2-pyrrolidinecarboxylic acid, (S)-Pyroglutamic acid, 2-L-Pyrrolidone-5-carboxylic acid, 2-Pyrrolidinone-5-carboxylic acid, 5-Carboxy-2-pyrrolidinone, 5-Oxo-L-proline, 5-Oxoproline, 5-Pyrrolidinone-2-carboxylic acid, Glutimic acid, Glutiminic acid, L-2-Pyrrolidone-5-carboxylic acid, L-5-Carboxy-2-pyrrolidinone, L-5-Oxo-2-pyrrolidinecarboxylic acid, L-5-Oxoproline, L-Glutamic acid, gamma-lactam, L-Glutimic acid, L-Glutiminic acid, L-Pyroglutamic acid, L-Pyrrolidinonecarboxylic acid, L-Pyrrolidonecarboxylic acid, Oxoproline, PCA, Pidolic acid, Pyroglutamic acid, Pyrrolidinonecarboxylic acid, Pyrrolidone-5-carboxylic acid, and Pyrrolidonecarboxylic acid.

The DL form of pyroglutamic acid (a mixture of the D and L stereoisomers) is known by the following names: DL-Proline, 5-oxo-(.+−.)-2-Pyrrolidone-5-carboxylic acid, (.+−.)-Pyroglutamic acid, 5-Oxo-DL-proline, DL-2-Pyrrolidinone-5-carboxylic acid, DL-2-Pyrrolidone-5-carboxylic acid, DL-Pyroglutamate, DL-Pyroglutamic acid, DL-Pyrrolidonecarboxylic acid, and Oxoproline. The DL form is also commercially available from Ajinomoto under the tradenames Ajidew A 100 and Ajidew N 50 (Na—PCA).

Some of the above-listed stereoisomers are commercially available from UCIB, France via Barnet Products Corp., New Jersey. Such compounds are sold under trade names like Cuivridone (Cu—PCA) and L-FER Pidolate (Fe—PCA), and Pidolidone.

### Metal Compound

The methods of the present invention include the administration of respiratory tract compositions that comprise a metal compound in combination with the organic acid described hereinabove. The respiratory tract compositions are administered to areas of the upper respiratory tract such as the nasal cavity to prevent and treat symptoms associated with respiratory viral infections, particularly SARS.

The concentration of the metal compound in the respiratory tract compositions of the present invention typically ranges from about 0.001% to about 20%, preferably from about 0.01% to about 10%, more preferably from about 0.05% to about 5%, most preferably from about 0.05% to about 2%, by weight of the composition. The metal compound can be included in the respiratory tract compositions as an individual metal compound or as a combination of metal compounds, wherein the total concentration of metal compound typically ranges from about 0.001% to about 20% by weight of the composition.

As previously stated, the respiratory tract compositions of the present invention can comprise pyroglutamic acid in combination with a monocarboxylic, dicarboxylic acid, or tricarboxylic acid defined herein. Preferably, the respiratory tract compositions comprise a metal compound, pyroglutamic acid, and a monocarboxylic, dicarboxylic acid, or tricarboxylic acid. Without being limited by theory, it is believed that when the respiratory tract compositions of the present invention include the metal compound, pyroglutamic acid, and monocarboxylic, dicarboxylic acid, or tricarboxylic acid, the metal compound and pyroglutamic acid can form a metal-acid complex that can provide for a synergistic immediate and residual anti-viral effect. The metal compound can be combined with pyroglutamic acid to form a metal-acid complex prior to incorporation of the metal-acid complex into the respiratory tract compositions of the present invention. If the metal-acid complex is formed prior to inclusion in the respiratory tract compositions herein, the metal-acid complex is included at concentrations ranging from about 0.001% to about 20%, preferably from about 0.01% to about 10%, more preferably from about 0.1% to about 5%, by weight of the composition. It is also contemplated that the respiratory tract compositions of the present invention can comprise the combination of metal compound and pyroglutamic acid without the inclusion of an additional organic acid.

Nonlimiting examples of metal compounds suitable for use herein include those metal compounds commonly referred to as “metal salts” which comprise metal ion substituents selected from the group consisting of manganese (Mn), silver (Ag), zinc (Zn), tin (Sn), iron (Fe), copper (Cu), aluminum (Al), nickel (Ni), cobalt (Co), and mixtures thereof. Preferred metal compounds include those metal compounds which contain Cu, Fe, or Zn metal ions, or combinations thereof. Examples of such metal compounds include the metal compounds referred to as salicylates, fumarates, benzoates, glutarates, lactates, citrates, malonates, acetates, glycolates, thiosalicylates, adipates, succinates, gluconates, aspartates, glycinates, tartarates, malates, maleates, ascorbates, chlorides, sulphates, nitrates, phosphates, fluorides, iodides, pidolates, and mixtures thereof. The acetates, ascorbates, chlorides, benzoates, citrates, gluconates, glutarates, lactates, malates, malonates, salicylates, succinates, sulphates, and mixtures thereof are preferred metal compounds.

Specific examples of a metal compound suitable for use herein include zinc acetate, zinc chloride, zinc ascorbate, zinc gluconate, zinc pidolate, zinc succinate, zinc sulphate, zinc chloride, and mixtures thereof. Zinc acetate is the most preferred metal compound.

When the respiratory tract compositions of the present invention comprise a metal compound containing zinc ion, it is believed that the zinc ion provides for antiviral properties. Furthermore, it is known that metal ions such as iron, silver, copper, and zinc can provide antiviral properties for the prevention and treatment of respiratory tract viral infections symptomatic of the common cold and influenza. Particularly, zinc and its possible effects on common colds has been extensively documented, The Handbook for Curing the Common Cold, George A. Eby, published 1994, George Eby Research, Texas, USA. It has been found, however, that respiratory tract compositions that comprise a metal compound containing zinc are also effective in preventing and treating symptoms associated with SARS.

### Pharmaceutically Acceptable Vehicle

The respiratory tract compositions of the present invention are typically administered to respiratory tract areas as formulations comprising a pharmaceutically acceptable vehicle or carrier system. Any pharmaceutically acceptable vehicle in the form of a liquid, solid, or gas is suitable for the delivery of the respiratory tract compositions to prevent and treat respiratory tract viral infections, particularly, SARS.

Depending on the desired form and delivery device to be used, the respiratory tract compositions of the present invention can be combined with pharmaceutically acceptable vehicles such as water, water-miscible solvents including ethanol, propylene glycol, polyethylene glycol, transcutol, glycerol, and other known or otherwise effective water-miscible solvents;

liquid aerosol propellants; and mixtures thereof. Preferably these vehicles are isotonic with human plasma.

When the respiratory tract compositions are administered using water as a pharmaceutically acceptable vehicle, the water is preferably purified or de-ionized water and is free of organic impurities. The concentration of water utilized to formulate the respiratory tract compositions into a final product form for delivery to respiratory tract areas ranges from about 40% to about 99.98%, preferably from about 80% to about 99.95%, by weight of the final product formulation.

When the respiratory tract compositions of the present invention are administered using a solid pharmaceutically acceptable vehicle, the vehicle may be applied in a powder form. In other words, the respiratory tract compositions of the present invention can be applied as a solid powder containing the essential ingredients and any optional components described herein with or without any known or otherwise effective solidification aids. However, pharmaceutically acceptable solid vehicles can be added to provide aid in processing of the compositions, to aid in the consistency of the compositions, to provide for improved stability, to facilitate handling, for hygroscopicity benefits, and so forth. Pharmaceutically acceptable solid vehicle materials include ingredients such as particulate and powder fillers, for example, a lactose powder. For respiratory tract compositions in the form of nasal compositions that are administered using a solid powder pharmaceutically acceptable vehicle, the particle size of the powder is typically greater than 10 microns, especially when the nasal composition is a nasal inhalant.

### Optional Components

The respiratory tract compositions of the present invention may further comprise one or more optional components known or otherwise effective for use in pharmaceutical compositions, provided that the optional components are physically and chemically compatible with the essential organic acid and metal compound components described hereinabove, or do not otherwise unduly impair product stability, aesthetics, or performance. Optional components suitable for use herein include materials such as mucoadhesive polymers, pH adjusting agents, chelating agents, preservatives, sensates, sweeteners, flavors, volatile oils, mucilages, surfactant spreading aids including polyoxyethylene (20) sorbitan mono-oleate commercially sold as Polysorbate 80, and so forth. The optional components can be included in the respiratory tract compositions at concentrations ranging from about 0.001% to about 30%, preferably from about 0.01% to about 10%, by weight of the composition.

The respiratory tract compositions of the present invention can optionally comprise homeopathic ingredients. A detailed, but not necessarily a complete list, of such homeopathic ingredients is found in The Homeopathic Pharmacopoeia of the United States, 1999 ed., published by The Pharmacopoeia Convention of the American Institute of Homeopathy, ©1982, Vol. 1-4, which descriptions are incorporated herein by reference. Specific nonlimiting examples of known, homeopathic, or otherwise effective, optional components suitable for use herein are described in more detail hereinbelow.

Optional Mucoadhesive Polymer

The respiratory tract compositions of the present invention preferably comprise an optional mucoadhesive polymer that provides for improved retention of the compositions in areas of the respiratory tract such as the nasal cavity to result in improved prevention and treatment of respiratory tract viral infections such as SARS without causing nasal irritation. It is known that mucoadhesive polymers can be incorporated into respiratory tract compositions such as nasal compositions to exhibit stimulus responsiveness. By stimulus responsiveness, it is meant that upon contacting the mucosal fluids or tissues, the compositions become sufficiently tacky or viscous to adhere to the tissues and do not quickly erode from the surface.

The optional mucoadhesive polymer suitable for use herein includes any solid or liquid used alone or in combination to impart a change in the viscosity of the respiratory tract compositions upon contact of the compositions with stimulus such as pH, body temperature, change in ionic concentration, and the like. Therefore, mucoadhesive polymers are sometimes commonly referred to as viscosity building polymers. It has been found that the incorporation of a mucoadhesive polymer that provides for a change in viscosity results in reducing and/or eliminating perceived irritation, especially perceived nasal irritation, that can be caused by the acidic nature of the respiratory tract compositions of the present invention.

The optional mucoadhesive polymer suitable for use herein provides for the adherence of the respiratory tract compositions to mucosal tissues, particularly nasal mucosal tissues, such that the acidic respiratory tract compositions comes into contact with mucosal tissues and fluids to result in a change in viscosity of the compositions in the respiratory tract area. As a result, the respiratory tract compositions of the present invention are maintained on the mucosal surface for periods longer than typical respiratory tract compositions, thus maintaining a virus-hostile environment for the improved prevention and treatment of respiratory tract viral infections. For example, if the respiratory tract compositions of the present invention are administered as a liquid respiratory tract composition, the composition is applied using an atomizing sprayer, and upon spraying into a respiratory tract area such as the nasal cavity the composition quickly forms a polymeric film, preferably a polymeric viscous film, that adheres to the nasal tissues. The polymeric film is preferably a thin polymer film, more preferably a thin polymeric viscous film, that is also resistance to erosion upon sneezing, blowing of the nose, or upon mucociliary clearance. The optional mucoadhesive polymer is also capable of changing the viscosity of the compositions in situ upon application of the respiratory tract compositions to the mucosal tissues and fluids.

The optional mucoadhesive polymer can be included in the respiratory tract compositions of the present invention as an individual mucoadhesive polymer or as a combination of mucoadhesive polymers, provided that the total concentration of mucoadhesive polymer ranges from about 0.01% to about 30%, preferably from about 0.1% to about 20%, more preferably from about 1% to about 15%, by weight of the composition.

The incorporation of the optional mucoadhesive polymer into the respiratory tract compositions of the present invention typically results in a composition that has a viscosity in the range of from about 1 centipoise (cps) to about 2000 cps, preferably from about 1 cps to about 1000 cps, more preferably from about 5 cps to about 500 cps, most preferably from about 5 cps to about 300 cps. The viscosity of the compositions can be measured by any known or otherwise effective technique employed to determine viscosity. Generally, the viscosity of the respiratory tract compositions of the present invention is determined using known methods such as those described in ASTM D 1824-87, ASTM D 1084-88, and ASTM D2196-86. Typical viscometers employed to measure viscosity include the Brookfield Syncho-Lectric Viscometer and the Haake Viscometer. For example, when the Brookfield Syncho-Lectric Viscometer is utilized for viscosity measurements, this viscometer is typically equipped with a spindle 4 to measure viscosities of less than 8,000 centipoise at low shear rates at given rotational speeds. Likewise, when the Haake Viscometer is utilized, a suitable Haake Viscometer is the Rheostress 1 model that is equipped with a probe (i.e., spindle) such as probe C35/2T wherein the viscosity measurement is performed over a temperature range of 5° C. to 40° C. at 50 revolutions per minute (rpm)/second (sec).

Known optional mucoadhesive polymers suitable for use herein are selected from the group consisting of carboxypolymethylenes, carboxyvinyl polymers, homopolymers of acrylic acid crosslinked with an allyl ether of pentaerythritol, homopolymers of acrylic acid crosslinked with an allyl ether of sucrose, homopolymers of acrylic acid crosslinked with divinyl glycol, and mixtures thereof.

Nonlimiting examples of suitable homopolymers of acrylic acid crosslinked with an allyl ether of pentaerythritol or an allyl ether of sucrose are available from B. F. Goodrich Company under the tradename “Carbopol”. Specific Carbopols include Carbopol 934, 940, 941, 956, 980, and mixtures thereof. Carbopol 980 is preferred among the carbopol mucoadhesive polymers. Polymers of this type have slightly acidic carboxyl group substituents. Such polymers generally have a pH of around 3 in water and are generally used by neutralization during preparation of compositions to form viscous films and/or gels. When the respiratory tract compositions of the present invention comprise one or more optional Carpobol mucoadhesive polymers, generally these polymers are used at concentrations ranging from about 0.01% to about 2.5% by weight of the composition.

Nonlimiting examples of suitable homopolymers of acrylic acid crosslinked with divinyl glycol are available from B. F. Goodrich Company as polycarbophils under the tradename “Noveon.”

Other nonlimiting examples of an optional mucoadhesive polymer suitable for use herein include natural polymers, polymeric cellulose derivatives, polyvinyl pyrrolidones (PVPs), dextran polymers, polyethylene oxide polymers including Polyox-600, thermoreversible polymers, ionic responsive polymers, copolymers of polymethyl vinyl ether and maleic anhydride, and mixtures thereof. Polymeric cellulose derivatives and thermoreversible polymers are preferred.

Specific nonlimiting examples of natural polymers suitable for use as an optional mucoadhesive polymer herein include arabic gums, tragacanth gums, agar polymers, xanthan gums, copolymers of alginic acid and sodium alginate, chitosan polymers, pectins, carageenans, pullulan polymers, modified starches, and mixtures thereof.

Specific nonlimiting examples of polymeric cellulose derivatives suitable for use as a preferred optional mucoadhesive polymer herein include hydroxy alkyl cellulose polymers including hydroxypropyl methylcellulose (HPMC) and hydroxypropyl cellulose (HPC), methyl cellulose polymers, carboxymethyl cellulose (CMC) polymers, salts of carboxymethyl cellulose including sodium salt of carboxymethyl cellulose, and mixtures thereof.

Specific nonlimiting examples of thermoreversible polymers suitable for use as a preferred optional mucoadhesive polymer herein include poloxamers including those poloxamers sold under the Lutrol F-127 and Lutrol F-68 tradenames, ethylhydroxy ethylcellulose (EHEC), and mixtures thereof.

Specific nonlimiting examples of ionic responsive polymers suitable for use as an optional mucoadhesive polymer herein include gelrite, gellan gum, Kelcogel F, and mixtures thereof.

Specific nonlimiting examples of copolymers of polymethyl vinyl ether and maleic anhydride suitable for use as an optional mucoadhesive polymer herein include such copolymers sold under the Gantrez tradename including Gantrez S and Gantrez MS type copolymers.

The optional mucoadhesive polymer suitable for use herein is more fully described in the Journal Pharmacy Pharmacology 53, pages 3-22, (2001 Edition); the International Journal of Pharmaceutics (1988, 1996 and 1998 Editions); and the Journal Controlled Release 62, pages 101-107, (1999 Edition); which descriptions are incorporated herein by reference.

Optional pH Adjusting Agent

The respiratory tract compositions of the present invention can optionally comprise pH adjusting agents. Optional pH adjusting agents can be included in the respiratory tract compositions of the present invention to adjust the pH of the compositions to values less than about 4.5. Therefore, when the compositions are applied to respiratory tract areas such as nasal tissues, the pH of the composition on the nasal tissues remains from about 3.0 to about 5.5, but is not so low as to cause irritation of the nasal tissues. Such optional pH adjusting agents include those normally associated with use in nasal compositions including compounds such as sodium bicarbonate, sodium phosphate, sodium hydroxide, ammonium hydroxide, triethanolamine, sodium citrate, disodium succinate, and mixtures thereof. If present, the optional pH adjusting agents are generally included at concentrations ranging from about 0.01% to about 5.0% by weight of the composition.

Optional Chelating Agents

The respiratory tract compositions of the present invention can optionally comprise chelating agents which are believed to provide for enhanced antiviral activity. Optional chelating agents useful in the respiratory tract compositions of the present invention include those that chelate transition metal ions such as iron, copper, zinc and other such metals. Not to be bound by theory, it is reasonable to postulate that metal ions, specifically metal cations, play a major role in the formation of oxidizing species. Oxidizing reactions and free radical formation can contribute to cellular damage in inflammatory diseases. The optional chelating agents useful herein are known to dampen oxidation reactions. The optional chelating agents are stable and effective in non-aqueous and aqueous mediums, and at pH ranges between about 3 to about 6. Nonlimiting examples of suitable optional chelating agents include phytic acid, disodium and calcium salts of ethylene diamine tetraacetic acid (EDTA), tetrasodium EDTA, sodium hexametaphosphate (SHMP), di(hydroxyethyl)glycine, 8-hydroxyquinoline, and mixtures thereof.

If the respiratory tract compositions of the present invention comprise one or more optional chelating agents, the chelating agents are included at concentrations ranging from about 0.001% to 10.00%, preferably from about 0.005% to about 5.0%, more preferably from about 0.01% to about 2%, by weight of the composition.

Optional Preservatives

The respiratory tract compositions of the present invention can optionally comprise preservatives. Preservatives can optionally be included to prevent microbial contamination that can be attributed to dosing devices or the respiratory tract composition applied to the nose. Such optional preservatives include those normally associated with use in nasal compositions including benzalkonium chloride, chlorhexidine gluconate, p enyl ethyl alcohol, phenoxyethanol, benzyl alcohol, sorbic acid, thimerosal, phenylmercuric acetate, and mixtures thereof.

### Method of Manufacture

The respiratory tract compositions of the present invention may be prepared by any known or otherwise effective technique suitable for providing a pharmaceutical composition that provides a therapeutic benefit in the prevention and treatment of respiratory tract viral infections. The respiratory tract compositions are preferably formulated to comprise the metal compound, organic acid, and optional mucoadhesive polymer described herein, wherein these compositions are then manufactured into final product forms of liquids, sprays, powders, inhalants, pumps, drops, and so forth for administration to respiratory areas to prevent and treat symptoms due to respiratory tract viral infections, particularly SARS.

When the respiratory tract compositions are administered using a pharmaceutically acceptable vehicle such as a liquid to deliver the compositions in product forms of sprays, pumps, droplets, and the like, the respiratory tract compositions are generally prepared by solubilizing an optional mucoadhesive polymer in a liquid vehicle such as water. While stirring, pyroglutamic acid if desired, a metal compound, and an organic acid are then added to the polymer solution. Next, a sensate mix is added while the solution is allowed to continue stirring. The sensate mix is typically added as a premix solution that can contain a combination of ingredients such as a combination of ethanol, menthol, peppermint oil, and spearmint oil. The pH of the resultant product should be between about 3.0 and about 5.5, however, a pH adjusting agent such as sodium hydroxide and/or disodium succinate can be added to maintain the pH of the resultant product to values less than about 4.5. These respiratory tract compositions administered in their liquid final product forms are especially suitable for incorporation into fill dropper vials, wherein the compositions are sprayed into a respiratory tract areas such as the nostrils or turbinates for effective prevention and treatment of respiratory tract viral infections. Typically, about 1 microliter (μl) to about 500 microliters (μls) are sprayed into each nostril or turbinate.

When the respiratory tract compositions of the present invention are administered using a pharmaceutically acceptable vehicle such as a powder, the compositions are generally prepared by dry blending pyroglutamic acid if desired, a metal compound, and an organic acid using a V-mixer. A pH adjusting agent such as sodium citrate can be added to the dry blend. The dry blend is then micronized using a fluid energy mill. The resultant micronized dry blend is then dry mixed with a powder filler such as lactose powder. This final powder respiratory tract composition can optionally be coated with a sensate premix using known spray coating techniques. The final powder respiratory tract composition can be filled into a nasal inhalation metering pump to prevent and treat symptoms associated with respiratory tract viral infections, wherein about 10 milligrams (mgs) of the final powder can be administered to a respiratory tract area such as a nostril or a turbinate.

As stated herein, the respiratory tract compositions of the present invention are suitable for administration in final product forms of liquids, sprays, pumps, inhalants, powders, and so forth. Suitable devices utilized in the administration of these final respiratory tract compositions include those commonly employed or otherwise effective liquid containers, droppers, spray containers including pressurized sprayers, pump containers, inhalation devices, powder containers, atomizers, and so forth.

### Method of Use

The present invention is directed to methods of preventing and treating respiratory tract viral infections by administering compositions described herein to respiratory tract areas such as the nasal cavity. Generally, a safe and effective amount of the compositions is applied to the respiratory tract area, particularly the nasal cavity. In this context, the term “safe and effective amount” refers to an amount which provides a therapeutic benefit with minimal or no adverse reactions.

As referred to herein, the methods of preventing and treating respiratory tract viral infections include any known or otherwise effective method of preventing and treating viruses and/or viral strains that can affect the respiratory tract to result in symptoms associated with the common cold, influenza, and SARS.

To prevent and treat respiratory tract viral infections, a safe and effective amount of the compositions of the present invention are administered to the respiratory tract. The safe and effective amount will depend on factors such as the type of composition administered, for example, the compositions of the present invention can be administered using product forms such as liquids, sprays, powders, inhalants, pumps, drops, and the like.

A preferred method of preventing and treating respiratory tract viral infections involves spraying the compositions of the present invention into the nasal cavity. For respiratory tract compositions in the form of nasal sprays, effective amounts of from about 1 microliter to about 500 microliters, preferably from about 1 microliter to about 150 microliters, are sprayed into each nostril or turbinate of the nasal cavity one or more times to administer an effective method of preventing and treating respiratory tract viral infections. Typically, about 50 microliters of the nasal spray is administered two to three times into each nostril or turbinate as an effective method of preventing and treating respiratory tract viral infections. For respiratory tract compositions in the form of diluted nasal sprays or nasal irrigations, from about 0.1 milliliters (mls) to about 50 milliliters are sprayed into each nostril or turbinate one or more times. It has been found that upon spraying the compositions into the nasal cavity, the infectious viruses and/or viral strains are effectively treated to alleviate symptoms associated with respiratory tract viral infections, particularly SARS, in addition to preventing the reoccurrence of virus symptoms.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. All exemplified concentrations are weight-weight percents, unless otherwise specified.

Exemplary respiratory tract compositions of the present invention are exemplified in Table II hereinbelow. These respiratory tract compositions preferably comprise a sensate premix exemplified in Table I hereinbelow. The exemplified sensate premixes of Table I provide for respiratory tract compositions that are aesthetically pleasing in taste, flavor, coolness, smell, and the like.

The respiratory tract compositions exemplified hereinbelow in Table II are suitable for spraying into respiratory tract areas such as the nostrils or turbinates for effective prevention and treatment of respiratory tract viral infections. Typically, about 1 microliter to about 500 microliters of the composition are sprayed into each nostril or turbinate.

TABLE I

| | Sensate Mix A | Sensate Mix B | Sensate Mix C |
|---|---|---|---|
| Component | (Wt. %) | (Wt. %) | (Wt. %) |
| Ethanol | 47.16 | — | — |
| Menthol | 29.41 | 11.565 | 51.38 |
| Peppermint Oil | 17.61 | — | — |
| Spearmint Oil | 5.82 | — | — |
| Phenyl Ethyl Alcohol | — | 77.495 | — |
| Camphor | — | 6.971 | 31.19 |
| Eucalyptol | — | 3.969 | 17.43 |
| Total: | 100 | 100 | 100 |

TABLE II

| | Sample 1 | Sample 2 | Sample 3 | Sample 4 | Sample 5 | Sample 6 |
|---|---|---|---|---|---|---|
| Component | (Wt. %) | (Wt. %) | (Wt. %) | (Wt. %) | (Wt. %) | (Wt. %) |
| Pyroglutamic Acid¹ | 0.35 | 0.70 | 1.00 | 0.37 | 1.70 | 0.35 |
| Succinic Acid² | 1.00 | 0.70 | 0.35 | 1.05 | 1.35 | 1.00 |
| Zinc Acetate Dihydrate³ | 0.12 | 0.012 | 0.12 | 0.12 | 0.12 | 0.12 |
| Zinc-EDTA⁴ | 0.255 | 0.255 | 0.255 | 0.255 | 0.255 | 0.255 |
| Polysorbate 80 | 0.05 | 0.05 | 0.05 | 0.12 | 0.08 | 0.08 |
| Carbopol 980⁵ | — | — | 1.20 | — | — | — |
| Hydroxypropyl methyl cellulose⁶ | 1.20 | — | — | 1.05 | 0.80 | 1.00 |
| Lutrol F-127⁷ | — | 15.8 | — | — | — | — |
| Sodium saccharin | — | 0.025 | 0.025 | 0.026 | — | 0.025 |
| Sucralose | 0.025 | — | — | — | 0.02 | — |
| Phenyl ethyl alcohol | 0.37 | 0.37 | 0.35 | — | 0.25 | 0.325 |
| Sodium chloride | 0.20 | 0.20 | 0.50 | 0.21 | 0.20 | — |
| Sensate Mix A | 0.067 | — | — | 0.45 | — | — |
| Sensate Mix B | — | 0.49 | 0.45 | — | 0.55 | — |
| Sensate Mix C | — | — | — | — | — | 0.109 |
| Sodium hydroxide (30%) | — | — | 0.10 | — | — | — |
| Disodium succinate | 1.00 | 0.50 | — | 0.46 | 0.25 | 0.44 |
| Propylene glycol | — | — | — | 0.52 | 0.25 | — |
| Propyl gallate | — | — | — | 0.01 | 0.02 | — |
| Deionized Water | q.s. 100 | q.s. 100 | q.s. 100 | q.s. 100 | q.s. 100 | q.s. 100 |

Wt. % - weight percent
¹pyroglutamic acid available from UCIB, France via Barnet Products Corp., New Jersey
²succinic acid available from DSM Fine Chemicals, UK
³zinc acetate dihydrate available from Verdugt B. V., Belgium
⁴zinc-EDTA available from Akzo Nobel Functional Chemicals bv, The Netherlands
⁵Carbopol 980 available from B. F. Goodrich Company, USA.
⁶hydroxypropyl methylcellulose available from Colorcon Ltd, Kent, UK
⁷Lutrol F-127 available from BASF Speciality Chemicals, Mount Oliver, NJ, USA

While particular embodiments suitable for use in the method of the present invention have been described, it will be obvious to those skilled in the art that various changes and modifications of the present invention can be made without departing from the spirit and scope of the invention. It is intended to cover, in the appended claims, all such modifications that are within the scope of this invention.

What is claimed is:

1. A method of preventing and treating SARS by administering a respiratory tract composition having a pH of from about 3.0 to about 5.5 to areas of the upper respiratory tract, wherein the respiratory tract composition comprises:
(a) from about 0.001% to about 20% by weight of an organic acid; and
(b) from about 0.01% to about 20% by weight of a metal compound.

2. The method of claim 1 wherein the organic acid is selected from the group consisting of ascorbic acid, salicylic acid, fumaric acid, benzoic acid, glutaric acid, lactic acid, citric acid, malonic acid, acetic acid, glycolic acid, malic acid, adipic acid, succinic acid, aspartic acid, phthalic acid, tartaric acid, glutamic acid, gluconic acid, pyroglutamic acid, and mixtures thereof.

3. The method of claim 1 wherein the metal compound is selected from the group consisting of salicylates, fumarates, benzoates, glutarates, lactates, citrates, malonates, acetates, glycolates, thiosalicylates, adipates, succinates, gluconates, aspartates, glycinates, tartarates, malates, maleates, ascorbates, chlorides, sulphates, nitrates, phosphates, fluorides, iodides, pidolates, and mixtures thereof.

4. The method of claim 3 wherein the metal compound is an acetate metal compound.

5. The method of claim 4 wherein the acetate is zinc acetate.

6. The method of claim 1 wherein the respiratory tract composition further comprises a mucoadhesive polymer selected from the group consisting of carboxypolymethylenes, carboxyvinyl polymers, homopolymers of acrylic acid crosslinked with an allyl ether of pentaerythritol, homopolymers of acrylic acid crosslinked with an allyl ether of sucrose, homopolymers of acrylic acid crosslinked with divinyl glycol, natural polymers, polymeric cellulose derivatives, polyvinyl pyrrolidones (PVPs), dextran polymers, polyethylene oxide polymers, thermoreversible polymers, ionic responsive polymers, copolymers of polymethyl vinyl ether and maleic anhydride, and mixtures thereof.

7. The method of claim 6 wherein the respiratory tract composition has a viscosity of from about 1 cps to about 2000 cps.

8. The method of claim 7 wherein the mucoadhesive polymer is a cellulose derivative selected from the group consisting of hydroxypropyl methylcelluloses, hydroxypropyl celluloses, methyl cellulose polymers, carboxymethyl cellulose polymers, salts of carboxymethyl cellulose, and mixtures thereof.

9. The method of claim 7 wherein the mucoadhesive polymer is a thermoreversible polymer selected from the group consisting of poloxamers, ethylhydroxy ethylcelluloses, and mixtures thereof.

10. The method of claim 6 wherein the respiratory tract composition further comprises a pH adjusting agent selected from the group consisting of sodium bicarbonate, sodium phosphate, sodium hydroxide, ammonium hydroxide, sodium stannate, triethanolamine, sodium citrate, disodium succinate, and mixtures thereof.

11. The method of claim 1 wherein the respiratory tract composition is a nasal composition.

12. The method of claim 11 wherein the nasal composition is selected from the group consisting of nasal liquids, nasal sprays, nasal inhalants, nasal powders, nasal drops, nasal irrigations, and mixtures thereof.

13. The method of claim 12 wherein the nasal composition is a nasal spray.

14. The method of claim 13 wherein the nasal spray comprises from about 40% to about 99.98% by weight of a pharmaceutically acceptable vehicle selected from the group consisting of water, ethanol, propylene glycol, polyethylene glycol, transcutol, glycerol, a liquid aerosol propellant, and mixtures thereof.

15. The method of claim 14 wherein the nasal spray contacts mucosal tissue and fluid.
